(19) United States
(12) Patent Application Publication
(10) Pub. No.: US 2024/0050081 A1
(43) Pub. Date: Feb. 15, 2024

(54) Surgical Tip Retrac Vac - Y

(71) Applicant: Nelson Jose Finol Gutierrez, Tampa, FL (US)

(72) Inventor: Nelson Jose Finol Gutierrez, Tampa, FL (US)

(21) Appl. No.: 17/819,935

(22) Filed: Aug. 15, 2022

(51) Int. Cl. A61B 17/02

(52) U.S. Cl. CPC A61B 17/0218; A61B 2217/005

(57) ABSTRACT

The present invention of utility consists of a device that has multiple uses and functionality since it allows the Retraction of tissues, structures, or organs and at the same time the Suction of fluids, being an advantage the maneuvering of this device with only one hand, which simplifies surgical procedures. This device is called Surgical Tip Retract Vac Y, an instrument made of metallic materials, plastic among others according to its usefulness and longevity, it is made up of a triangular and/or pyramidal shape with blunt edges, equidistant sides joined to a flat surface or base of retraction making an ergonomic grip and simple to use. Suction is provided through the main hole located in the suction tip with multiple accessory suction holes in the area to avoid collapse of the device during its functionality.

### FIELD OF THE INVENTION

The present invention is related to a device useful in the medical-surgical field, the utility consists on the retraction of tissue and anatomical structures and in turn control of fluids through the suction. Its use is essential in specialties such as Dentistry, Otorhinolaryngology, plastic surgery, neurosurgery and among others that merit the use of suction and retraction tips.

### BACKGROUND

In surgical procedures it is important to note that there is a variety of actions that are essential to obtain a correct and precise visualization of the area where the procedure is being performed, among these actions are the following: retraction of anatomical structures, this consists in the persistent reduction of the dimensional volume of the tissue, and the control of fluids that is managed with the suction, which consists of the force that a partial vacuum exerts on a liquid, gas, etc. The functions of tissue retraction and fluid suction are essential for any operative and surgical procedure.

The retraction of the tissues is carried out through different devices with flat, semi-flat, concave surfaces, among others that allow the displacement of the structures prior to the application of a counterforce by the operator or assistants of a certain procedure. The control of the fluids is determined by means of the suction of the same, such as: blood, saline solution among other liquid substances used during surgical procedures for the irrigation of mechanical, rotary, surgical cutting devices of mini-dimensional size frequently used for the surgical excision or removal of any pathology in a person or individual. The function of retraction and suction are essential in the operative procedures, these are done singularly or separately with different devices using one at a time for each of the hands of the operator or assistants in any case.

On the market there are a variety of devices used for suction during medical surgical procedures. The different ejectors or surgical suction tips are determined by a variety of uses and operative purposes, which we can mention Frazier cannula, Yankawuer cannula, surgical dental tip or ejector, saliva ejector, among others. They can have different diameters, sizes and are composed for different materials such as metal, PVC or plastics, the mentioned above are characterized by being disposable or single use.

Currently the drawbacks that the operator faces when approaching a surgical procedure is the visualization of the operative field either by the presence of fluids or tissues in the area to be treated. For this, the assistant or surgical operator assists with suction, which has a limited function because it does not have the retraction function.

The suction tips or the active suction part of the device are used through the mobility provided by the assistant or surgeon, and this is done by sucking in a prudent and continuous way the fluids or secretions from wounds or organic and inorganic tissues, which can be present during the surgery, this manipulation being usually ineffective when performing the execution of psychomotor maneuvers by the participants at the time of a certain procedure.

The use of suction and retraction devices are essential to optimize the procedure, by allowing the visualization of the tissues and/or anatomical structures of the human body, this visualization is the result of the aspiration of fluids during the surgical procedure with the purpose of cleaning and visualization.

Tissue retraction is usually done by the operator or assistant with one hand while suction devices are usually used by the assistant with the opposite hand. Therefore, the suction of fluids and retraction of the tissues simultaneously are performed with a single hand individually, which makes the procedure delay and not very dynamic.

### BRIEF SUMMARY OF THE INVENTION

The present invention comprises a device called Surgical Tip Retrac Vac-Y, this consists of an instrument that has the function of retracting the tissues or anatomical structures simultaneously with the suction of the fluids, being manageable with a single hand by the operator or assistants in a certain procedure.

This is made up of different parts that form its external and internal structure, all assembled, making it a single piece from its initial end (suction tip) to its terminal end (vacuum connector or connection end).

This device consists of an external area and an internal area, the external area is made up of: Aspiration Tip, Support Rod, Connection End, Connector Top and Retraction Surface. The internal area is made up of a conical cylindrical shape, the circumference of the terminal end being greater diameter than the initial end, the internal surface is smooth from its initial part to its terminal part.

### BRIEF DESCRIPTION OF THE DRAWINGS

FIG. 1. Anterolateral View. The external area constituted by the front-end support rod (lateral portion of the suction tip) and rear end (lateral portion of the connector and top of the connector) is shown, delimited by its semi-triangular shape where one of its sides is evidenced.

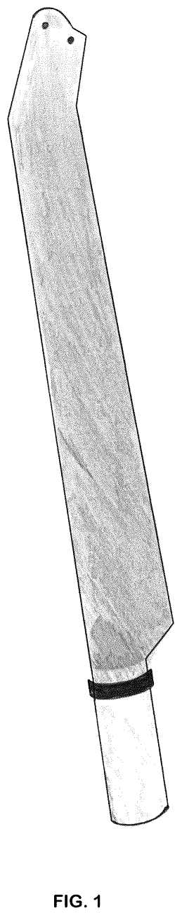

FIG. 2. Upper Postero View. In this view, the rear end (connection end and connector stop) is detailed in these structures with a continuous surface to the support rod determined by their triangular shape with a convex surface on the upper surface of the support rod.

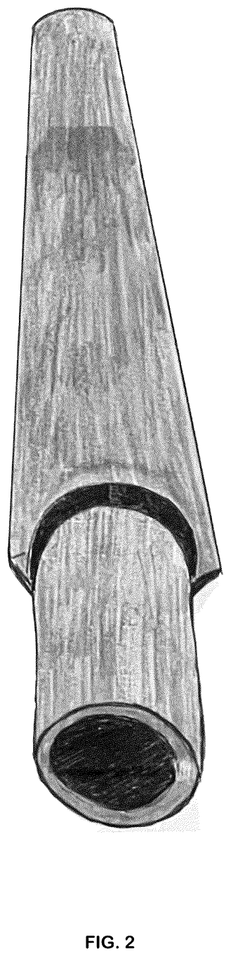

FIG. 3. Postero-Lateral View, in this view, the support rod delimited by the anterior end (Aspiration Tip) is detailed, posterior end (Connector area and Connector Top), also the lateral area of the retraction surface is visualized.

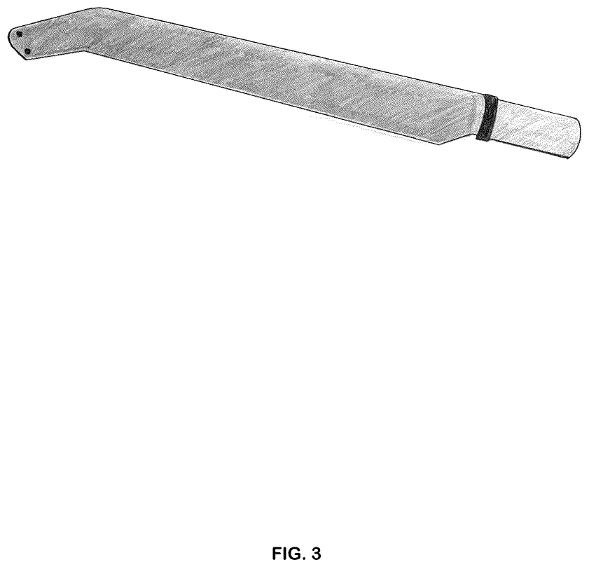

FIG. 4. Anterior Inferior Lateral View. In this view, the anterior surface of the Aspiration Tip, determined by its pyramidal and triangular shape, is visualized. A convex surface is also evidenced in the extension of the anterior end that corresponds to the support rod that presents a lateral area, and a flat zone that corresponds to the retraction surface is also observed.

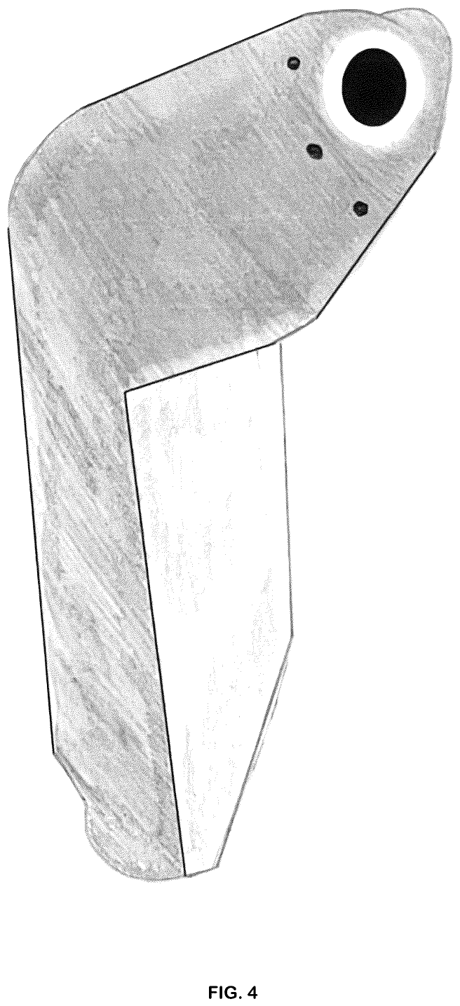

FIG. 5. Previous View. The anterior end of the device designated as the aspiration tip is shown, the anterior hole formed by its triangular shape, whose lateral slopes join a convex line, evidenced by a blunt and smooth upper edge that extends and continues through the support rod, is shown the lower edge of the retraction surface.

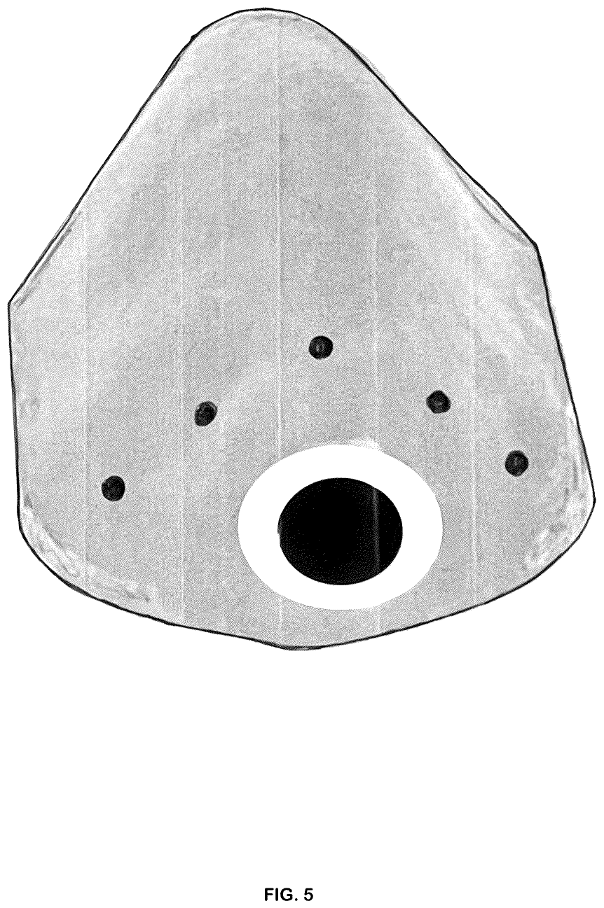

FIG. 6. Bottom View. The retraction surface is evidenced by determining the angled anterior part in its proper portion (suction tip) and the posterior area determined by the connector, the lower surface shows the anterior part.

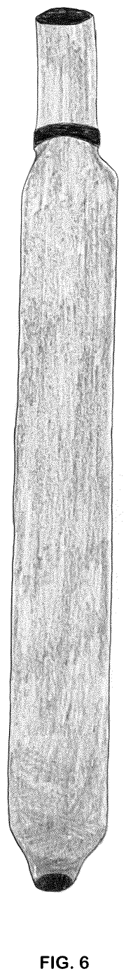

### DETAILED DESCRIPTION OF THE INVENTION

In this invention a utility device called Surgical Tip Retrac Vac-Y is detailed. This consists of a multifunctional instrument that allows several actions to be carried out at the same time, simplifying procedures and psychomotor activities by a specific individual. The main function is to retract the tissues and/or anatomical structures and allow the suction of fluids, thus contributing to the specific visualization of surgically intervened areas in a given individual. The advantage of this device is that its use does not require bimanual use and it is manageable with only one hand, making this highly relevant during surgical maneuvers, allowing the operator or assistant to add maneuvers or mobility of other devices typical of any surgical procedure.

This device can be presented in various materials, whether metallic, plastic, among others. Due to its use, it has a specific utility on the part of the operator, who will select the material according to the nature of the surgical procedure. This device is made up of an external and internal area.

The external area is composed of the following parts: Surgical Suction Tip, This area can be described as follows:
Upper Antero Zone: consists of the anterior area of the suction point angled or perpendicular in relation to the longitudinal arrangement of the support rod composed of two slopes with a triangular and conical shape whose vertex converge both slopes constituting the back of the suction point smooth blunt and with a continuous surface throughout. This tip has accessory suction holes on the sides of it which are circular in shape.
Lower Posterior Zone: it is a smooth and flat surface with a triangular and conical shape whose slopes converge on the contour of the anterior suction hole.
Lateral borders: they make up the convergence zone of the upper antero area with the lower postero area with a rectangular shape at its origin and continued by a triangular shape delimited by the anterior border.
Front edge: made up of a ring or circumference.

Support Rod: it is made up of a conical cylindrical upper surface and a flat lower surface, both extended from the aspiration tip to the vacuum connector. For a more detailed description of the stem, we decided to divide it into lateral areas, upper and lower area and lateral borders.

1. (canceled)

2. (canceled)

3. (canceled)

4. (canceled)

5. (canceled)

6. (canceled)

7. (canceled)

8. (canceled)

9. A Surgical Tip Retract Vac-Y comprising: an aspiration tip, a support rod, a connection end, a connector top and a retraction surface.

10. A Surgical Tip Retract Vac-Y as in claim 1, wherein said an aspiration tip is angled relative to main axis of the support rod.

11. A Surgical Tip Retract Vac-Y as in claim 1, wherein said an aspiration tip have holes around the cylinder surface.

12. A Surgical Tip Retract Vac-Y as in claim 1, wherein said a support rod present triangular and pyramidal shape.

13. A Surgical Tip Retract Vac-Y as in claim 1, wherein said a retraction surface is flat, triangular anterior area and rectangular middle portion.

14. A Surgical Tip Retract Vac-Y comprising use for retraction, protection, and suction simultaneously.

15. A Surgical Tip Retract Vac-Y as in claim 2, wherein said comprising use for retraction of tissues and anatomical structures and get better visualization.

16. A Surgical Tip Retract Vac-Y as in claim 2, wherein said comprising use for protection of tissues an anatomical structure.

17. A Surgical Tip Retract Vac-Y as in claim 2, wherein said comprising use for suction of liquids and fluids.
